(19) United States
(12) Patent Application Publication
(10) Pub. No.: US 2026/0217628 A1
(43) Pub. Date: Jul. 30, 2026

(54) *MRE-based catalysts for olefin skeletal isomerization

(71) Applicants: Sina Sartipi, Willebroek (BE); Marc H. Anthonis, Hofstade (BE)

(72) Inventors: Sina Sartipi, Willebroek (BE); Marc H. Anthonis, Hofstade (BE)

(21) Appl. No.: 19/143,025

(22) PCT Filed: Dec. 21, 2023
(86) PCT No.: PCT/EP2023/087394

§ 371 Date: Jun. 25, 2025

(30) Foreign Application Priority Data
Jan. 18, 2023 (EP) 23152249.1

(51) Int. Cl. C07C 5/22; B01J 29/70

(52) U.S. Cl. CPC C07C 5/222; B01J 29/703; C07C 2529/70

(57) ABSTRACT

A method for isomerizing alpha olefins to produce an isomerization mixture comprising branched olefins can comprise contacting an olefinic feed including one or more C10-C20 alpha olefins with a Kaolin *MRE catalyst under skeletal isomerization conditions, wherein the Kaolin *MRE catalyst is a synthesized molecular sieve having an *MRE topography, wherein in the synthesis thereof at least one source of alumina is a Kaolin family clay; and obtaining an isomerization mixture comprising one or more C10-C20 branched olefins.

### CROSS-REFERENCE TO RELATED APPLICATIONS

This application is related to WO2022/103468 entitled “Method of Synthesizing Aluminum-Rich Molecular Sieve of *MRE Framework Type.”

### FIELD

This application relates to methods for *MRE-based skeletal isomerization to produce an isomerization mixture comprising branched olefins.

### BACKGROUND

Branched olefins are commercially valuable for use in a variety of applications such as intermediates in the manufacture of industrial fluids, cleaning agents, and solvent products. For example, branched olefins may be hydrotreated to provide fluids exhibiting a number of desirable properties, such as biodegradability and low viscosity and/or hydroformylated to provide alcohol products.

Branched olefins may be produced via the skeletal isomerization of alpha olefins, preferably linear alpha olefins. Skeletal isomerization is used to manufacture higher branched olefins with higher octane numbers from linear olefins. Olefin skeletal isomerization over zeolites, both natural and synthetic, has demonstrated effectiveness for the catalytic conversion of petroleum streams. One type of zeolite structure for olefin skeletal isomerization is ZSM-48 which has orthorhombic or pseudo-orthorhombic symmetry and ten-ring non-interconnecting, linear channels whose ideal dimensions are 5.5×5.6 Å. The framework structure of ZSM-48 has been assigned the three-letter code *MRE. According to R. F. Lobo et al. (J. Am. Chem. Soc., 2002, 124, 13222-13230), ZSM-48 is not a code for one material but for a family of materials with different degrees of disorder. As a result, molecular sieves of the *MRE framework type can comprise zeolites of the ZSM-48 family such as at least one of COK-8, EU-2, EU-11, IZM-1, SSZ-91, ZBM-30, and ZSM-48.

However, olefin skeletal isomerization over zeolites may be accompanied by molecular weight growth side reactions. Such side reactions can create undesirable byproducts (i.e., C₂₀₊ branched olefins) that are considered yield loss and often require separation, resulting in at least increased economic and processing time costs.

Accordingly, there is a need for highly active and selective methods of isomerizing alpha olefins to branched olefins that minimizes undesirable side reaction byproducts and thus maximizes olefin skeletal isomerization.

### SUMMARY

This application relates to methods for *MRE-based skeletal isomerization to produce an isomerization mixture comprising branched olefins.

In one or more aspects of the present disclosure, a method is provided comprising contacting an olefinic feed comprising one or more C₁₀-C₂₀ alpha olefins with a Kaolin *MRE catalyst under skeletal isomerization conditions, wherein the Kaolin *MRE catalyst is a synthesized molecular sieve having an *MRE topography, wherein in the synthesis thereof at least one source of alumina is a Kaolin family clay; and obtaining an isomerization mixture comprising one or more C₁₀-C₂₀ branched olefins.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, as will occur to one of ordinary skill in the art and having the benefit of this application.

FIG. 1 is a bar graph showing skeletal isomerization results of the zeolite comparative examples comprising crystals (Catalyst A, Example 1) v. formulations (Catalyst B, Example 2; Catalyst C, Example 3; Catalyst D, Example 4 (Catalyst C with lower loading and flow rate, as described below).

FIG. 2 is a bar graph showing skeletal isomerization results of the zeolite crystals of sodium aluminate comparative examples (Catalyst A, Example 1; Catalyst E, Example 5; Catalyst G, Example 7) v. Kaolin experimental example (Catalyst H, Example 8).

FIG. 3 is a bar graph showing skeletal isomerization results of the zeolite crystals of Kaolin experimental examples (Catalyst H, Example 8; Catalyst I, Example 9; Catalyst J, Example 10; Catalyst K, Example 11; Catalyst L, Example 12) assessing the influence of Si/Al₂ ratios.

FIGS. 4A and 4B are charts showing the on-stream stability of skeletal isomerization results of the zeolite crystals of sodium aluminate comparative example (Catalyst G, Example 7) v. Kaolin experimental example (Catalyst J, Example 10) at various temperatures.

FIGS. 5A and 5B are olefin chromatograms showing skeletal isomerization results of the zeolite crystals of sodium aluminate comparative example (Catalyst G, Example 7) v. Kaolin experimental example (Catalyst J, Example 10) at various temperatures.

### DETAILED DESCRIPTION

This application relates to methods for *MRE-based skeletal isomerization to produce an isomerization mixture comprising branched olefins.

These methods require an olefinic feed including one or more C₁₀-C₂₀ alpha olefins, preferably linear alpha olefins, and a catalyst to provide mixtures having C₁₀-C₂₀ branched olefins.

The methods described herein use catalysts including molecular sieves having an *MRE topology, as described in WO 2022/103468, the entirety of which is incorporated herein by reference. As used herein, the terms “Kaolin *MRE catalyst” and “Kaolin *MRE” are defined as synthesized molecular sieves having an *MRE topography, wherein in the synthesis thereof at least one source of alumina (herein also referred to as a precursor) is a clay selected from the Kaolin family. The Kaolin *MRE catalysts may comprise precursors including an alumina source of Kaolin, at least one source of silica, at least one source of hydroxide ions, at least one source of alkali and/or alkaline earth metal M, at least one source of linear diquaternary alkylammonium structure directing agent R, water, and optional seed crystals. Upon synthesis, the Kaolin *MRE catalysts comprise the following molar ratio composition: SiO₂:Al₂O₃ (or simply Si:Al₂) from 15 to less than 100, OH—:SiO₂ from 0.02 to 0.8, M:SiO₂ from 0.02 to 0.8, R:SiO₂ from 0.005 to 0.5, and H₂O:SiO₂ from 5 to 100, where the source of the alumina is Kaolin (added during synthesis in solid form).

Kaolin maybe in the form of any Kaolin family member, and are commonly known as Dixie, McNamee, Georgia and Florida clays, or others in which the main mineral constituent is kaolinite, halloysite, dickite, nacrite, or anauxite. Kaolinite, halloysite, dickite and nacrite are polymorph clay minerals with the empirical formula Al₂Si₂O₅(OH)₄. Anauxite is considered a mixture of kaolinite and free silica. Metakaolin is obtained by dehydration of kaolin clays such as from kaolinite and can be described by formula Al₂Si₂O₇.

The Kaolin *MRE catalysts for olefin skeletal isomerization, as provided herein, demonstrate minimized undesirable molecular weight growth side reaction byproducts compared to conventional zeolite *MRE catalysts. As used herein, the terms “conventional zeolite *MRE catalyst” or “conventional *MRE catalyst” are defined as synthesized molecular sieves having an *MRE topography, wherein in the synthesis thereof the source of alumina does not comprise a clay from the Kaolin family. In particular examples, the “conventional zeolite *MRE catalyst” is a synthesized molecular sieve having an *MRE topography, wherein in the synthesis thereof the source of alumina (precursor) is sodium aluminate. Accordingly, the Kaolin *MRE catalysts of the present disclosure demonstrate comparatively improved olefin skeletal isomerization yield.

### Definitions

All numerical values within the detailed description and the claims herein are modified by “about” or “approximately” the indicated value, and take into account experimental error and variations that would be expected by a person having ordinary skill in the art.

As used herein, “wt %” means percentage by weight, “mol %” means percentage by mole, “ppm” means parts per million, and “ppmw” means parts per million on a weight basis. All “ppm” as used herein are ppm by weight unless specified otherwise. All concentrations herein are expressed on the basis of the total amount of the composition in question. Thus, the concentrations of the various components of the first mixture are expressed based on the total weight of the first mixture. All ranges expressed herein should include both end points as two specific embodiments unless specified or indicated to the contrary.

As provided above, and as used herein, the terms “Kaolin *MRE catalyst” and “Kaolin *MRE” are defined as synthesized molecular sieves having an *MRE topography, wherein in the synthesis thereof at least one source of alumina (herein also referred to as a precursor) is a clay selected from the Kaolin family.

As provided above, and as used herein, the term “conventional zeolite *MRE catalyst” or “conventional *MRE catalyst” are defined as synthesized molecular sieves having an *MRE topography, wherein in the synthesis thereof the source of alumina does not comprise a clay from the Kaolin family. In particular examples, the “conventional zeolite *MRE catalyst” is a synthesized molecular sieve having an *MRE topography, wherein in the synthesis thereof the source of alumina (precursor) is sodium aluminate.

The term “hydrocarbon” means a class of compounds containing hydrogen bound to carbon, and encompasses (i) saturated hydrocarbon compounds; (ii) unsaturated hydrocarbon compounds; and (iii) mixtures of hydrocarbon compounds (saturated and/or unsaturated), including mixtures of hydrocarbon compounds having different values of n (i.e., differing carbon numbers).

As used herein, a “carbon number” refers to the number of carbon atoms in a hydrocarbon. Likewise, a “Cₓ” hydrocarbon is one having x carbon atoms (i.e., carbon number of x), and a “Cₓ-C_{y}” or “C_{x-y}” hydrocarbon is one having from x to y carbon atoms.

The term “olefin,” alternatively referred to as “alkene,” refers to a branched or unbranched unsaturated hydrocarbon having one or more carbon-carbon double bonds. A simple olefin comprises the general formula CₙH₂ₙ, where n is 2 or greater. Examples of olefins include, but are not limited to, ethylene, propylene, butylene, pentene, hexene, and heptene. “Olefin” is intended to embrace all structural isomeric forms of an olefin. For example, butylene encompasses but-1-ene, (Z)-but-2-ene, and the like.

As used herein, the term “molecular sieve” is used synonymously with the term “zeolite” or “microporous crystalline material.”

As used herein, the term “reactor” refers to any vessel(s) in which a chemical reaction occurs. Reactor includes both distinct reactors, as well as reaction zones within a single reactor apparatus and, as applicable, reaction zones across multiple reactors. For example, a single reactor may have multiple reaction zones. Where the description refers to a first and second reactor, the person of ordinary skill in the art will readily recognize such reference includes two reactors, as well as a single reactor vessel having first and second reaction zones. Likewise, a first reactor effluent and a second reactor effluent will be recognized to include the effluent from the first reaction zone and the second reaction zone of a single reactor, respectively.

Various embodiments described herein provide processes for the production of one or more C₁₀-C₂₀ branched olefins via skeletal isomerization (the single term “isomerization” may be used interchangeably with “skeletal isomerization” herein) of one or more C₁₀-C₂₀ linear alpha olefins (LAOs). It has been found that employing a Kaolin *MRE catalyst for olefin skeletal isomerization allows for minimization of undesirable molecular weight growth side reaction byproducts (e.g., C₂₀₊ branched olefins)

It has been found that employing molecular sieve catalysts having a *MRE topology in skeletal isomerization advantageously allows for the isomerization to be conducted under mild process conditions. Additionally, conducting the isomerization at a relatively elevated temperature over Kaolin *MRE provides several benefits, including improving selectivity to desired products in the resulting isomerization mixture and reducing undesired products. Additionally, it has been found that such catalysts are particularly effective in controlling branched olefin formation in the produced isomerization mixture. Generally, the resulting isomerization mixture comprises a minimized linear olefin content in a range low enough to maintain biodegradability properties of the mixture while high enough to maintain an acceptably low pour point. For example, the isomerization of the C₁₀-C₂₀ alpha olefinic feed using a Kaolin *MRE catalyst results in a conversion of the C₁₀-C₂₀ alpha olefins in a range from about 20% to about 100%, such as from about 20% to about 95%, or from about 40% to about 90%, from about 50% to about 85%, or from about 60% to about 80%.

### Olefinic Feed

Generally, the LAOs supplied for skeletal isomerization have a carbon number ranging from 10 to 20, more preferably from 12 to 18, more preferably from 12 to 16, and ideally from 12 to 14.

Typically, the one or more C₁₀-C₂₀ alpha olefins are provided in an olefinic feed. Suitable olefinic feeds for use in various embodiments of the present disclosure comprise (or consist essentially of, or consist of) C₁₀-C₂₀ alpha olefins, preferably C₁₂-C₁₈ alpha olefins, such as C₁₂-C₁₆ alpha olefins, ideally C₁₂-C₁₄ alpha olefins. In any one or more aspects, at least about 50 wt %, preferably at least about 60 wt %, more preferably at least about 80 wt %, more preferably at least about 85 wt %, more preferably at least about 95 wt %, and more preferably at least about 99 wt % (including up to 100 wt %) of the olefinic feed is composed of alpha olefins, preferably linear alpha olefins, having any of the aforementioned Cₓ-C_{y} ranges (i.e., any of the aforementioned numbers of carbon atoms) based on the total weight of the olefinic feed. For example, in any one or more aspects the olefinic feed may comprise from about 40 wt % to 100 wt %, such as from about 75 wt % to about 90 wt %, of alpha olefins, preferably linear alpha olefins, having any of the aforementioned Cₓ-C_{y} ranges based on the total weight of the olefinic feed. Particularly preferable olefinic feeds may comprise C₁₂-C₁₆ alpha olefins, ideally C₁₂-C₁₄ linear alpha olefin mixtures. In any one or more aspects, the olefinic feed typically comprises at least about 40 wt % of C₁₄ alpha olefins, more preferably at least about 60 wt %, such as at least about 65 wt % of C₁₄ alpha olefins (preferably linear C₁₄ alpha olefins) based on the total weight of the olefinic feed and, additionally or alternatively, at most about 60 wt %, more preferably at most about 40 wt %, such as at most about 35 wt % of C₁₄ alpha olefins (preferably linear C₁₄ alpha olefins) based on the total weight of the olefinic feed, such as from about 60 wt % or from about 65 wt % to 75 wt % C₁₄ alpha olefins and from about 25 wt % to about 40 wt % or to about 35 wt % C₁₄ alpha olefins based on the total weight of the olefinic feed.

In any embodiment, the olefinic feed preferably has an average carbon number (by weight, as measured by GC-MS) of greater than or equal to 12, preferably less than or equal to 16, such as from 12 to 16.

Typically, the olefinic feed is substantially or wholly linear. For example, the olefinic feed typically has a branched olefin content of less than 10 wt % based on the total weight of the olefinic feed, preferably less than about 8 wt %, more preferably less than about 4 wt %, such as from 0 wt % to 10 wt % branched olefin content based on the total weight of the olefinic feed.

Preferably, the olefinic feed is pretreated prior to skeletal isomerization to remove moisture, oxygenates, nitrates, and other impurities that could deactivate the Kaolin *MRE catalyst of the present disclosure. Typically, the pretreatment is performed by passing the olefinic feed through a guard bed that contains a sieve. Typically, the pretreated feed comprises less than about 50 ppmw water based on the weight of the feed, more preferably less than about 25 ppmw.

### Kaolin *MRE Catalyst

As used herein, molecular sieves of the *MRE framework type comprise zeolites of the ZSM-48 family such as at least one of COK-8, EU-2, EU-11, IZM-1, SSZ-91, ZBM-30, and ZSM-48. In the following, the expressions “zeolite” and “molecular sieve” can be used interchangeably. Also, the expression “ZSM-48 family” is used synonymously with the expression “molecular sieve of *MRE framework type.” The term “ZSM-48 family” material as used herein, includes one or more of:
molecular sieves made from a common first degree crystalline building block unit cell, which unit cell has the *MRE framework topology (A unit cell is a spatial arrangement of atoms which if tiled in three-dimensional space describes the crystal structure. Such crystal structures are discussed in the “Atlas of Zeolite Framework Types,” Fifth edition, 2001, the entire content of which is incorporated as reference);
molecular sieves made from a common second degree building block, being a 2-dimensional tiling of such *MRE framework topology unit cells, forming a monolayer of one unit cell thickness, preferably one c-unit cell thickness;
molecular sieves made from common second degree building blocks, being layers of one or more than one unit cell thickness, wherein the layer of more than one unit cell thickness is made from stacking, packing, or binding at least two monolayers of one unit cell thickness. The stacking of such second degree building blocks can be in a regular fashion, an irregular fashion, a random fashion, or any combination thereof, and
molecular sieves made by any regular or random 2-dimensional or 3-dimensional combination of unit cells having the *MRE framework topology.

More particularly, molecular sieves of the *MRE framework type comprise a family of materials having straight channels (or tubular) pores. The pores are formed of rolled up honeycomb-like sheets of fused T6-rings (T=tetrahedral), and the pore aperture contains 10 T-atoms. Neighboring pores are related by a zero shift along the pore direction or by a shift of half the repeat distance along the pore direction. Molecular sieves of the *MRE framework type generally have an X-ray diffraction pattern including d-spacing maxima at 11.8±0.2, 10.2±0.2, 7.2±0.15, 4.2±0.08, 3.9±0.08, 3.6±0.06, 3.1±0.05 and 2.85±0.05 Angstrom. The X-ray diffraction data used to characterize the material are obtained by standard techniques using the K-alpha doublet of copper as the incident radiation and a diffractometer equipped with a scintillation counter and associated computer as the collection system.

The method of synthesizing aluminum-rich molecular sieve of *MRE framework type according to the present disclosure is described in WO 2022/103468, the entirety of which is incorporated herein by reference, and involves preparing a synthesis mixture according to conventional techniques, except that the synthesis mixture comprises a source of alumina in the form of Kaolin having a low water solubility. Preferably, the synthesis mixture only comprises a Kaolin source of alumina having a low water solubility. As used herein, the expression “low water solubility” means alumina in the form of Kaolin that is not readily soluble in a synthesis mixture at room temperature (e.g., about 20° C.) and near-neutral pH (e.g., about pH 7) and that is not dissolved prior to its addition into the synthesis mixture (e.g., in a caustic solution. The method of synthesizing a molecular sieve according to the disclosure further involves crystallizing the molecular sieve according to conventional techniques, and isolating the molecular sieve according to conventional techniques.

The suitable source of alumina having a low water solubility includes clays selected from Kaolin families. Typical examples of appropriate solid form include powder form, for instance solid particles having a mean particle size of from about 0.01 μm to about 300 μm, such as from 0.1 μm to 200 μm or from 1 μm to 100 μm, as volume weighted mean measured by dynamic light scattering (DLS). Clays selected from kaolin families are commonly known as Dixie, McNamee, Georgia and Florida clays, or others in which the main mineral constituent is kaolinite, halloysite, dickite, nacrite, or anauxite. Kaolinite, halloysite, dickite and nacrite are polymorph clay minerals with the empirical formula Al₂Si₂O₅(OH)₄. Anauxite is considered as a mixture of kaolinite and free silica. Metakaolin is obtained by dehydration of kaolin clays such as from kaolinite and can be described by formula Al₂Si₂O₇.

While other, typical, sources of alumina might also be present in the synthesis mixture, (i.e., sources of alumina that are readily soluble in the synthesis mixture or in the form of a solution for instance in caustic solution), such typical sources of alumina should only be present in minor amounts, that is 10 mol % or less of the total sources of alumina in terms of total Al₂O₃ amount, in particular 5 mol % or less, more particularly 2 mol % or less. In preferred aspects, the synthesis mixture contains essentially no additional sources of alumina, except the Kaolin having a low water solubility as defined above.

The Kaolin *MRE catalysts comprise precursors including an alumina source of Kaolin, at least one source of silica, at least one source of hydroxide ions, at least one source of alkali and/or alkaline earth metal M, at least one source of linear diquaternary alkylammonium structure directing agent R, water, and optional seed crystals. Upon synthesis, the Kaolin *MRE catalysts comprise the following molar ratio composition: SiO₂:Al₂O₃ (or simply Si:Al₂) from 15 to less than 100, OH—:SiO₂ from 0.02 to 0.8, M:SiO₂ from 0.02 to 0.8, R:SiO₂ from 0.005 to 0.5, and H₂O:SiO₂ from 5 to 100, where the source of the alumina is Kaolin (added during synthesis in solid form).

The SiO₂:Al₂O₃ (or simply Si:Al₂) molar ratio in the Kaolin *MRE catalysts of the present disclosure in the synthesis mixture is typically at least 15, most often at least 20, in particular at least 25, such as at least 30, at least 40 or at least 50. For instance, the Si:Al₂ molar ratio in the synthesis mixture may be from 15 to less than 100, or from 20 to less than 100, or from 25 to less than 100, or from 30 to less than 100, in particular from 15, 20, 25, 30 or 40, 50, or 60 to less than 100, or to 60 to less than 90.

The molecular sieve may also be subjected to other treatments such as steaming and/or washing with solvent. Steaming, for example, can change the performance of catalyst/zeolite molecular sieve, depending on the particular application. Such treatments are well-known to the skilled person and are carried out in order to modify the properties of the molecular sieve as desired.

Once the Kaolin *MRE has been synthesized, it can be formulated into a product composition by combination with other materials, such as binders, surfactants, and/or matrix materials that provide additional hardness to the finished catalyst. These other materials can be inert or catalytically active materials. Calcination, ion-exchange, steaming, and/or washing can be performed on the as-synthesized Kaolin *MRE and/or after the Kaolin *MRE has been formulated into a product combination.

Alternately, the Kaolin *MRE catalyst may be substantially free of binder or surfactant, or free of binder or surfactant. Typically, the Kaolin *MRE catalyst is free or substantially free of additional components apart from the molecular, binder (if present), and optionally, trace amounts of alkali and/or alkali earth metals or compounds thereof. For example, in any one or more aspects of the present disclosure, the Kaolin *MRE catalyst may be free or substantially free from promoters, such as noble metals and transition metals in metal or metal oxide form (e.g., platinum, palladium, ruthenium, iron, cobalt, and nickel). For instance, preferably the Kaolin *MRE catalyst may comprise a combined platinum, palladium, ruthenium, iron, cobalt, and nickel content of less than about 0.5 wt % based on the weight of the Kaolin *MRE catalyst, more preferably less than about 0.1 wt % or less than about 0.01 wt %.

### Skeletal Isomerization of Olefins

The skeletal isomerization reaction can be conducted in a wide range of reactor configurations including fixed bed (single or in series) and fluidized bed, preferably fixed bed. In addition, the isomerization can be conducted in a single reaction zone or in a plurality of reaction zones.

Typically, the Kaolin *MRE skeletal isomerization is conducted under conditions suitable to maintain the reaction medium in the liquid phase. Suitable reaction temperatures range from at least about 160° C., such as from about 160° C. about 300° C., such as from about 170° C. to about 200° C., such as from about 180° C. to about 200° C., or from about 180° C. to about 300° C., or from about 150° C. to about 170° C., while suitable isomerization pressures range from about 0.0 barg to about 2 barg, or more preferably from about 0.5 barg to about 2 barg, or about 1 barg to about 2 barg. Preferably, the olefinic feed is supplied to the reaction at a weight hourly space velocity (WHSV) ranging from about 1 h⁻¹ to about 50 h⁻¹, more preferably from about 1 h⁻¹ to about 20 h⁻¹, more preferably from about 1 h⁻¹ to about 10 h⁻¹, wherein the WHSV is the weight of feed flowing per unit weight of the catalyst per hour. The temperature ranges may also vary with the activity loss of the catalyst (e.g., the temperature range may be increased to compensate for catalyst activity losses).

Typically, the Kaolin *MRE isomerization exhibits a high single-pass rate of conversion (measured as 100 minus the remaining amount of linear alpha olefins expressed in wt %, as measured by GC). For example, preferably the single-pass rate of conversion of the one or more C₁₀-C₂₀ alpha olefins is from about 5% to about 98%, more preferably from about 20% to about 98%. In such aspects, the isomerization can be conveniently conducted in the absence of recycle (i.e., without recycling any portion of the produced isomerization mixture). Preferably, conducting the isomerization without recycle provides several process advantages, such as increasing process reliability and reducing operating costs.

Preferably, the isomerization reaction is highly selective to the desired branched olefin products, and exhibits minimal side reactions, such as oligomerization and cracking. For example, typically less than about 30 wt % of C₁₀-C₂₀ alpha olefins present in the olefinic feed are converted to product having a lower or higher carbon number. Additionally or alternatively, typically less than about 30 wt % of linear C₁₀-C₂₀ alpha olefins present (if any) in the olefinic feed are converted to C₂₀₊ olefins.

### Isomerization Mixture

The resulting isomerization mixture obtained via Kaolin *MRE skeletal isomerization of the one or more C₁₀-C₂₀ alpha olefins according to any one or more of the foregoing aspects of the present disclosure typically comprises (or consists essentially of, or consists of) linear internal olefins, branched olefins (e.g., branched internal olefins), and, often, C₂₀₊ olefins. For example, the isomerization mixture preferably has a branched olefin content of from about 5 wt % to about 98 wt %, preferably from about 10 wt % to about 95 wt %, such as from about 20 wt % to about 90 wt %, or from about 25 wt % to about 85 wt % based on the total weight of the isomerization mixture. The isomerization mixture may further comprise from 0 wt % to about 90 wt %, preferably from about 10 wt % to about 85 wt %, more preferably from about 15 wt % to about 80 wt % of linear internal olefins based on the total weight of the isomerization mixture. The isomerization mixture may further comprise less than about 20 wt % of C₂₀₊ olefins or dimerized olefins, such as less than about 15 wt %, or less than about 10 wt %, or less than about 7 wt % of C₂₀₊ olefins or dimerized olefins by total weight of the isomerization mixture.

The Kaolin *MRE isomerization product may also contain some amount of residual C₁₀-C₂₀ alpha olefins. Preferably, the isomerization mixture comprises a residual C₁₀-C₂₀ alpha olefin content of less than about 35 wt %. In a first alternative, the isomerization mixture contains less than about 10 wt %, and ideally less than about 5 wt %, such as less than about 3 wt % of residual C₁₀-C₂₀ alpha olefins based on the total weight of the isomerization mixture. In a second alternative, the isomerization mixture contains from about 10 wt % to about 30 wt %, preferably from about 10 wt % to about 20 wt %, and ideally from about 10 wt % to about 15 wt %, of residual C₁₀-C₂₀ alpha olefins based on the total weight of the isomerization mixture.

In the present methods, it is found that molecular sieves having the Kaolin *MRE topology are particularly active for the skeletal isomerization of C₁₀-C₂₀ linear alpha olefins. In addition, such catalysts exhibit improved selectivity towards branched internal olefins while also providing improved control of C₂₀₊ olefin formation.

For example, in a first alternative, using a molecular sieve having a Kaolin *MRE topology comprising the isomerization of a C₁₄ linear alpha olefin feed, it is found that the resulting isomerization mixture may comprise:
from about 5 wt % to about 98 wt % of branched olefins, such as from 10 wt % to about 95 wt %, such as from about 15 wt % to about 90 wt %, or from about 20 wt % to about 85 wt %;
from 0 wt % to about 90 wt %, such as 10 wt % to about 85 wt %, for example from about 15 wt % to about 80 wt % of branched olefins;
less than about 20 wt % of C₂₀₊ olefins, such as C₂₈₊ olefins, such as less than about 15 wt %, or less than about 10 wt %, or less than about 7 wt % of C₂₀₊ olefins, such as C₂₈₊ olefins; and
less than about 10 wt %, such as less than about 5 wt %, for example less than about 3 wt % of linear alpha olefins.

Accordingly, in one or more aspects, olefinic mixtures produced in accordance with the processes of the present disclosure may advantageously comprise from about 5 wt % to about 98 wt % of branched olefins, such as from 10 wt % to about 95 wt %, such as from about 15 wt % to about 90 wt %, or from about 20 wt % to about 85 wt % branched olefins; from 0 wt % to about 90 wt %, such as 10 wt % to about 85 wt %, for example from about 15 wt % to about 80 wt % of branched olefins; less than about 20 wt % of C₂₀₊ olefins, such as C₂₈₊ olefins, such as less than about 15 wt %, or less than about 10 wt %, or less than about 7 wt % of C₂₀₊ olefins, such as C₂₈₊ olefins; and less than about 10 wt %, such as less than about 5 wt %, for example less than about 3 wt % of linear alpha olefins.

In a second alternative, using a molecular sieve having Kaolin *MRE topology in the isomerization of C₁₂ linear alpha olefin feed, it is found that the resulting isomerization mixture may comprise:
from about 5 wt % to about 98 wt % of branched olefins, such as from 10 wt % to about 95 wt %, such as from about 15 wt % to about 90 wt %, or from about 20 wt % to about 85 wt %;
from 0 wt % to about 90 wt %, such as 10 wt % to about 85 wt %, for example from about 15 wt % to about 80 wt % of branched olefins;
less than about 20 wt % of C₂₀₊ olefins, such as C₂₄₊ olefins such as less than about 15 wt %, or less than about 10 wt %, or less than about 7 wt % of C₂₄₊ olefins; and
less than about 10 wt %, such as less than about 5 wt %, for example less than about 3 wt % of linear alpha olefins.

Accordingly, in one or more aspects, olefinic mixtures produced in accordance with the methods of the present disclosure may advantageously comprise from about 5 wt % to about 98 wt % of branched olefins, such as from 10 wt % to about 95 wt %, such as from about 15 wt % to about 90 wt %, or from about 20 wt % to about 85 wt % branched olefins; from 0 wt % to about 90 wt %, such as 10 wt % to about 85 wt %, for example from about 15 wt % to about 80 wt % of branched olefins; less than about 20 wt % of C₂₀₊ olefins, such as C₂₄₊ olefins, such as less than about 15 wt %, or less than about 10 wt %, or less than about 7 wt % of C₂₀₊ olefins, such as C₂₄₊ olefins; and less than about 10 wt %, such as less than about 5 wt %, for example less than about 3 wt % of branched olefins.

In any one or all aspects, the branched olefins obtained in the isomerization mixture may be particularly useful as intermediates for hydrogenation for fluid applications and/or hydroformylation via the Oxo process for alcohol production. Preferred isomerization mixtures suitable for these applications may comprise 20 wt % or more of C₁₂-C₁₆ branched olefins. In a preferred embodiment, the isomerization mixture comprises, in a first alternative, about 20 wt % or more, in particular from about 5 wt % to about 98 wt % of C₁₂-C₁₆ branched olefins, such as from 10 wt % to about 95 wt %, such as from about 15 wt % to about 90 wt %, or from about 20 wt % to about 85 wt % C₁₂-C₁₆ branched olefins; from 0 wt % to about 90 wt %, such as 10 wt % to about 85 wt %, for example from about 15 wt % to about 80 wt % of C₁₂-C₁₆ branched olefins; less than about 20 wt % of C₂₀₊ olefins, such as less than about 15 wt %, or less than about 10 wt %, or less than about 7 wt % of C₂₀₊ olefins; and less than about 10 wt %, such as less than about 5 wt % of C₁₂-C₁₆ branched olefins.

### EXAMPLE EMBODIMENTS

Embodiments disclosed herein include:

Embodiment A: A method comprising contacting an olefinic feed comprising one or more C₁₀-C₂₀ alpha olefins with a Kaolin *MRE catalyst under skeletal isomerization conditions, wherein the Kaolin *MRE catalyst is a synthesized molecular sieve having an *MRE topography, wherein in the synthesis thereof at least one source of alumina is a Kaolin family clay; and obtaining an isomerization mixture comprising one or more C₁₀-C₂₀ branched olefins.

Embodiment A may have one or more of the following additional elements in any combination:

Element 1: wherein the Kaolin *MRE catalyst comprises a Si:Al2 molar ratio in the range of 15 to less than 100.

Element 2: wherein the Kaolin *MRE catalyst comprises a Si:Al2 molar ratio in the range of 50 to less than 100.

Element 3: wherein the Kaolin *MRE catalyst comprises a Si:Al2 molar ratio in the range of 60 to less than 90.

Element 4: wherein the Kaolin family clay is selected from the group consisting of halloysite, kaolinite, dickite, nacrite, anauxite, metakaolin, and any combination thereof.

Element 5: wherein the synthesized molecular sieve is a molecular sieve of the ZSM-48 family.

Element 6: wherein the Kaolin *MRE catalyst is free of noble metals and transition metals in metal or oxide form.

Element 7: wherein prior to contacting, forming the Kaolin *MRE catalyst into a product by combining the Kaolin *MRE catalyst with one or more of a binder, a surfactant, and/or a matrix material.

Element 8: wherein prior to contacting, performing one or more of calcination, ion-exchange, steaming, and/or washing the Kaolin *MRE catalyst.

Element 9: wherein the olefinic feed comprises one or more C12-C16 alpha olefins based on total weight of the olefinic feed.

Element 10: wherein the olefinic feed comprises one or more C12-C14 alpha olefins based on total weight of the olefinic feed.

Element 11: wherein the olefinic feed comprises at least 40 wt % of C14 alpha olefins based on total weight of the olefinic feed.

Element 12: wherein the olefinic feed comprises a branched olefin content in the range of 0 wt % to 10 wt % based on total weight of the olefinic feed.

Element 13: wherein prior to the contacting, pretreating the olefinic feed reduce water content thereof to a range of 50 ppmw water based on the weight of the olefinic feed.

Element 14: wherein the skeletal isomerization conditions comprise a temperature in the range of about 160° C. to about 300° C.

Element 15: wherein the skeletal isomerization conditions comprise a pressure in the range of 0 barg to 2 barg.

Element 16: wherein the skeletal isomerization conditions comprise contacting the olefin feed at a weight hourly space velocity in the range of from 1 h-1 to about 20 h-1 based on weight of the olefin feed flowing per unit of weight of the Kaolin *MRE catalyst per hour.

Element 17: wherein the conversion of the C10-C20 alpha olefins is from about 20% to about 98%.

Element 18: wherein the isomerization mixture comprises less than 20 wt % of C20+ olefins based on total weight of the isomerization mixture.

Element 19: wherein the isomerization mixture comprises 9 wt % to 90 wt % of linear internal olefins based on total weight of the isomerization mixture.

Embodiment A may have one or more of the following additional elements in any combination: 1 and any one, more, or all of 5-18 in any non-limiting combination; 2 and any one, more, or all of 5-18 in any non-limiting combination; 3 and any one, more, or all of 5-18 in any non-limiting combination.

To facilitate a better understanding of the embodiments described herein, the following examples of various representative embodiments are given. In no way should the following examples be read to limit, or to define, the scope of the present disclosure.

### EXAMPLES

In the following Examples, a series of catalysts based on ZSM-48 (*MRE topology) comprising alumina of either sodium aluminate (Conventional Zeolite *MRE Catalysts A-G) or Kaolin (Kaolin *MRE Catalysts H-L) were used in skeletal isomerization reactions of C₁₄ linear alpha olefins to C₁₄ branched olefins (desired product) and C₂₀₊ (undesired byproduct, as described above). Catalysts A-G comprising sodium aluminate represent comparative examples; Catalysts H-L comprising Kaolin represent experimental examples according to one or more aspects of the present disclosure.

The following Catalysts A-L were prepared using skeletal isomerization over various zeolite under varying temperature conditions, in which the temperature was increased or decreased in a step-wise fashion during the course of the isomerization run. The skeletal isomerization reactions of a C₁₄ linear alpha olefin feed were performed at 160-200° C. (note that two 160° C. reactions were performed to give an indication of catalyst deactivation) and 1.5 barg with WHSV set at 5 h⁻¹, set point based on liquid reactive feed and zeolite content of the catalyst; v=~7 cm² h⁻¹, aᵥ=4.5 cm² h⁻¹; m_{catalyst,target}=1 g (pure crystals), 1.54 g (formulated materials), a m_{catalyst,target}=1 g (formulated material); dₚ=250-300 μm, sized, where v refers to volumetric flow rate, aᵥ refers to the volumetric flow rate of Example 4, m refers to catalyst mass, am refers to the catalyst mass of Example 4, and dₚ refers to the catalyst particle size.

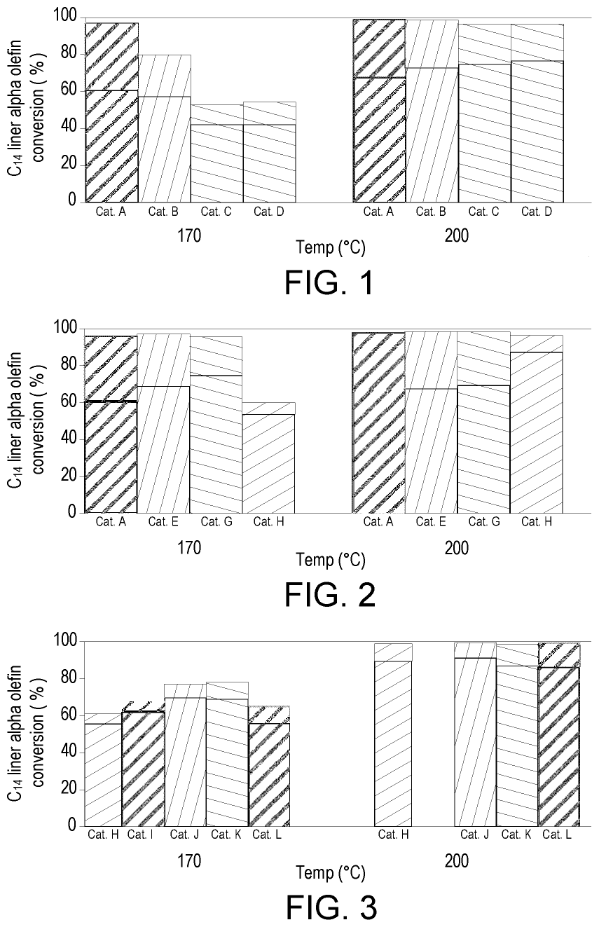

After describing each Catalyst (Examples 1-12), the results are described in FIGS. 1-5B (Examples 13-17) and conclusions provided.

### Example 1 (Comparative): Catalyst A (Sodium Aluminate Crystals—Si/Al₂=81)

Catalyst A is conventional zeolite *MRE catalyst; Si/Al₂=81 (as described above, this ratio is based on the synthesis gel). Catalyst A does not comprise binders and was not steamed. Skeletal isomerization was performed as described above, and the results of the yield percentage of C₁₄ branched olefins (Y_{C14} branched (%)) and the percentage of C₂₀₊ olefins (Y_{C20+}(%)) at varying temperatures is provided in Table 1, presented as average values after 100 hours on stream. It is to be noted that the percentage of C₁₄ branched olefins and the percentage of C₂₀₊ olefins may not equal 100%, as other linear or branched olefins (e.g., linear internal olefins due to double-bond shifting) may be present in the isomerization reaction product and other non-C₁₄ linear alpha olefins comprise a portion of the feed.

TABLE 1
Catalyst A

| Temp (° C.) | Y_{C14 branched} (%) | Y_{C20+} (%) |
|---|---|---|
| 160 | 55 | 33 |
| 170 | 60 | 37 |
| 180 | 59 | 39 |
| 190 | 65 | 35 |
| 200 | 67 | 33 |

### Example 2 (Comparative): Catalyst B (Sodium Aluminate Formulation—Si/Al₂=81)

Catalyst B is a conventional zeolite *MRE catalyst; Si/Al₂=81. Catalyst B is further formulated (compared to Catalyst A) with VERAL-300 (Honeywell ULP) alumina binder at 65% zeolite content, shaped to 1/20 inch quadralobe, pre-calcined, ion-exchanged, and again calcined. Skeletal isomerization was performed as described above, and the results of the yield percentage of C₁₄ branched olefins (Y_{C14} branched (%)) and the percentage of C₂₀₊ olefins (Y_{C20+}(%)) at varying temperatures is provided in Table 2, presented as average values after 100 hours on stream. It is to be noted that the percentage of C₁₄ branched olefins and the percentage of C₂₀₊ olefins may not equal 100%, as other linear or branched olefins (e.g., linear internal olefins due to double-bond shifting) may be present in the isomerization reaction product and other non-C₁₄ linear alpha olefins comprise a portion of the feed.

TABLE 2
Catalyst B

| Temp (° C.) | Y_{C14 branched} (%) | Y_{C20+} (%) |
|---|---|---|
| 160 | 38 | 16 |
| 170 | 56 | 24 |
| 180 | 67 | 29 |
| 190 | 68 | 31 |
| 200 | 72 | 27 |
| 160 | 24 | 4 |

### Example 3 (Comparative): Catalyst C (Sodium Aluminate Formulation—Si/Al₂=81)

Catalyst C is a conventional zeolite *MRE catalyst; Si/Al₂=81. Catalyst C is further formulated (compared to Catalyst A) with VERAL-300 alumina binder at 65% zeolite content, shaped to 1/16 inch quadralobe, pre-calcined, ion-exchanged, again calcined, and steamed for 3 hours at 371° C. Skeletal isomerization was performed as described above, and the results of the yield percentage of C₁₄ branched olefins (Y_{C14} branched (%)) and the percentage of C₂₀₊ olefins (Y_{C20+}(%)) at varying temperatures is provided in Table 3, presented as average values after 100 hours on stream. It is to be noted that the percentage of C14 branched olefins and the percentage of C20+ olefins may not equal 100%, as other linear or branched olefins (e.g., linear internal olefins due to double-bond shifting) may be present in the isomerization reaction product and other non-C₁₄ linear alpha olefins comprise a portion of the feed.

TABLE 3
Catalyst C

| Temp (° C.) | Y_{C14 branched} (%) | Y_{C20+} (%) |
|---|---|---|
| 160 | 19 | 7 |
| 170 | 41 | 12 |
| 180 | 67 | 19 |
| 190 | 74 | 23 |
| 200 | 74 | 23 |

### Example 4 (Comparative): Catalyst D (Sodium Aluminate Formulation—Si/Al₂=81)

Catalyst D is a conventional zeolite *MRE catalyst; Si/Al₂=81. Skeletal isomerization of Catalyst D was performed as described above, but with comparatively lower catalyst loading and flowrate (the WHSV remained the same; the loading and flowrate determined by v=4.5 cm²h⁻¹; m_{catalyst,target}=1 (formulated material)), and the results of the yield percentage of C₁₄ branched olefins (Y_{C14} branched (%)) and the percentage of C₂₀₊ olefins (Y_{C20+}(%)) at varying temperatures is provided in Table 4, presented as average values after 100 hours on stream. It is to be noted that Catalyst D is similar to Catalyst C, and was formulated (compared to Catalyst A) with VERAL-300 alumina binder at 65% zeolite content, shaped to 1/16 inch quadralobe, pre-calcined, ion-exchanged, again calcined, and steamed for 3 hours at 371° C. It is further to be noted that the percentage of C₁₄ branched olefins and the percentage of C₂₀₊ olefins may not equal 100%, as other linear or branched olefins (e.g., linear internal olefins due to double-bond shifting) may be present in the isomerization reaction yield and other non-C₁₄ linear alpha olefins comprise a portion of the feed.

TABLE 4
Catalyst D

| Temp (° C.) | Y_{C14 branched} (%) | Y_{C20+} (%) |
|---|---|---|
| 160 | 26 | 9 |
| 170 | 41 | 13 |
| 180 | 58 | 20 |
| 190 | 71 | 24 |
| 200 | 76 | 21 |

### Example 5 (Comparative): Catalyst E (Sodium Aluminate Crystals—Si/Al₂=100)

Catalyst E is a conventional zeolite *MRE catalyst; Si/Al₂=100. Catalyst E does not comprise binders and was not steamed. Skeletal isomerization of Catalyst E was performed as described above, and the results of the yield percentage of C₁₄ branched olefins (Y_{C14} branched (%)) and the percentage of C₂₀₊ olefins (Y_{C20+}(%)) at varying temperatures is provided in Table 5, presented as average values after 100 hours on stream. It is to be noted that the percentage of C₁₄ branched olefins and the percentage of C₂₀₊ olefins may not equal 100%, as other linear or branched olefins (e.g., linear internal olefins due to double-bond shifting) may be present in the isomerization reaction yield and other non-C₁₄ linear alpha olefins comprise a portion of the feed.

TABLE 5
Catalyst E

| Temp (° C.) | Y_{C14 branched} (%) | Y_{C20+} (%) |
|---|---|---|
| 160 | 68 | 27 |
| 170 | 69 | 30 |
| 180 | 73 | 26 |
| 190 | 71 | 28 |
| 200 | 67 | 32 |
| 160 | 79 | 7 |

### Example 6 (Comparative): Catalyst F (Sodium Aluminate Crystals—Si/Al₂=100)

Catalyst F is the conventional zeolite *MRE catalyst of Example 5; Si/Al₂=100. Catalyst F (compared to Catalyst E was prepared with cetrimonium bromide (CATB) added during synthesis. Skeletal isomerization of Catalyst F was performed as described above, and the results of the yield percentage of C₁₄ branched olefins (Y_{C14} branched (%)) and the percentage of C₂₀₊ olefins (Y_{C20+}(%)) at varying temperatures is provided in Table 6, presented as average values after 100 hours on stream. It is to be noted that the percentage of C₁₄ branched olefins and the percentage of C₂₀₊ olefins may not equal 100%, as other linear or branched olefins (e.g., linear internal olefins due to double-bond shifting) may be present in the isomerization reaction yield and other non-C₁₄ linear alpha olefins comprise a portion of the feed.

TABLE 6
Catalyst F

| Temp (° C.) | Y_{C14 branched} (%) | Y_{C20+} (%) |
|---|---|---|
| 160 | 60 | 35 |
| 170 | 68 | 28 |
| 180 | 67 | 32 |
| 190 | 68 | 31 |
| 200 | 67 | 33 |
| 160 | 82 | 8 |

### Example 7 (Comparative): Catalyst G (Sodium Aluminate Crystals—Si/Al₂=81)

Compared to Example 1, Catalyst G is made with synthesis duplication but at a smaller scale (affecting scale-dependent parameters e.g., mixing, heating, and the like).

Catalyst G is a conventional zeolite *MRE catalyst; Si/Al₂=81. Catalyst G does not comprise binders and was not steamed. Skeletal isomerization was performed as described above, and the results of the yield percentage of C₁₄ branched olefins (Y_{C14} branched (%)) and the percentage of C₂₀₊ olefins (Y_{C20+}(%)) at varying temperatures is provided in Table 7, presented as average values after 100 hours on stream. It is to be noted that the percentage of C₁₄ branched olefins and the percentage of C₂₀₊ olefins may not equal 100%, as other linear or branched olefins (e.g., linear internal olefins due to double-bond shifting) may be present in the isomerization reaction yield.

TABLE 7
Catalyst G

| Temp (° C.) | Y_{C14 branched} (%) | Y_{C20+} (%) |
|---|---|---|
| 160 | 57 | 18 |
| 170 | 74 | 22 |
| 180 | 74 | 25 |
| 190 | 71 | 28 |
| 200 | 69 | 31 |
| 160 | 63 | 5 |

### Example 8 (Experimental): Catalyst H (Kaolin Crystals—Si/Al₂=81)

Catalyst H is a Kaolin *MRE catalyst; Si/Al₂=81. Catalyst H does not comprise binders and was not steamed. Skeletal isomerization was performed as described above, and the results of the yield percentage of C₁₄ branched olefins (Y_{C14} branched (%)) and the percentage of C₂₀₊ olefins (Y_{C20+}(%)) at varying temperatures is provided in Table 8, presented as average values after 100 hours on stream. It is to be noted that the percentage of C₁₄ branched olefins and the percentage of C₂₀₊ olefins may not equal 100%, as other linear or branched olefins (e.g., linear internal olefins due to double-bond shifting) may be present in the isomerization reaction yield.

TABLE 8
Catalyst H

| Temp (° C.) | Y_{C14 branched} (%) | Y_{C20+} (%) |
|---|---|---|
| 160 | 39 | 6 |
| 170 | 53 | 6 |
| 180 | 75 | 8 |
| 190 | 86 | 9 |
| 200 | 87 | 10 |
| 160 | 19 | 4 |

### Example 9 (Experimental): Catalyst I (Kaolin Crystals—Si/Al₂=70)

Catalyst I is a Kaolin *MRE catalyst; Si/Al₂=70. Catalyst I does not comprise binders and was not steamed. Skeletal isomerization was performed as described above, and the results of the yield percentage of C₁₄ branched olefins (Y_{C14} branched (%)) and the percentage of C₂₀₊ olefins (Y_{C20+}(%)) at varying temperatures is provided in Table 9, presented as average values after 100 hours on stream. It is to be noted that the percentage of C₁₄ branched olefins and the percentage of C₂₀₊ olefins may not equal 100%, as other linear or branched olefins (e.g., linear internal olefins due to double-bond shifting) may be present in the isomerization reaction yield.

TABLE 9
Catalyst I

| Temp (° C.) | Y_{C14 branched} (%) | Y_{C20+} (%) |
|---|---|---|
| 160 | 31 | 4 |
| 170 | 60 | 6 |
| 180 | 82 | 9 |
| 190 | 87 | 10 |

### Example 10 (Experimental): Catalyst J (Kaolin Crystals—Si/Al₂=60)

Catalyst J is a Kaolin *MRE catalyst; Si/Al₂=60. Catalyst J does not comprise binders and was not steamed. Skeletal isomerization was performed as described above, and the results of the yield percentage of C₁₄ branched olefins (Y_{C14} branched (%)) and the percentage of C₂₀₊ olefins (Y_{C20+}(%)) at varying temperatures is provided in Table 10, presented as average values after 100 hours on stream. It is to be noted that the percentage of C₁₄ branched olefins and the percentage of C₂₀₊ olefins may not equal 100%, as other linear or branched olefins (e.g., linear internal olefins due to double-bond shifting) may be present in the isomerization reaction yield.

TABLE 10
Catalyst J

| Temp (° C.) | Y_{C14 branched} (%) | Y_{C20+} (%) |
|---|---|---|
| 160 | 43 | 6 |
| 170 | 67 | 9 |
| 180 | 84 | 6 |
| 190 | 88 | 8 |
| 200 | 89 | 9 |
| 160 | 39 | 0 |

### Example 11 (Experimental): Catalyst K (Kaolin Crystals—Si/Al₂=50)

Catalyst K is a Kaolin *MRE catalyst; Si/Al₂=50. Catalyst K does not comprise binders and was not steamed. Skeletal isomerization was performed as described above, and the results of the yield percentage of C₁₄ branched olefins (Y_{C14} branched (%)) and the percentage of C₂₀₊ olefins (Y_{C20+}(%)) at varying temperatures is provided in Table 11, presented as average values after 100 hours on stream. It is to be noted that the percentage of C₁₄ branched olefins and the percentage of C₂₀₊ olefins may not equal 100%, as other linear or branched olefins (e.g., linear internal olefins due to double-bond shifting) may be present in the isomerization reaction yield.

TABLE 11
Catalyst K

| Temp (° C.) | Y_{C14 branched} (%) | Y_{C20+} (%) |
|---|---|---|
| 160 | 47 | 9 |
| 170 | 67 | 11 |
| 180 | 80 | 11 |
| 190 | 84 | 11 |
| 200 | 85 | 13 |

### Example 12 (Experimental): Catalyst L (Kaolin Crystals—Si/Al₂=40)

Catalyst L is a Kaolin *MRE catalyst; Si/Al₂=40. Catalyst L does not comprise binders and was not steamed. Skeletal isomerization was performed as described above, and the results of the yield percentage of C₁₄ branched olefins (Y_{C14} branched (%)) and the percentage of C₂₀₊ olefins (Y_{C20+}(%)) at varying temperatures is provided in Table 12, presented as average values after 100 hours on stream. It is to be noted that the percentage of C₁₄ branched olefins and the percentage of C₂₀₊ olefins may not equal 100%, as other linear or branched olefins (e.g., linear internal olefins due to double-bond shifting) may be present in the isomerization reaction yield.

TABLE 12
Catalyst L

| Temp (° C.) | Y_{C14 branched} (%) | Y_{C20+} (%) |
|---|---|---|
| 160 | 33 | 8 |
| 170 | 54 | 10 |
| 180 | 73 | 11 |
| 190 | 84 | 13 |
| 200 | 84 | 14 |

### Example 13 (FIG. 1): Evaluation of Conventional *MRE Catalysts v. Kaolin *MRE Catalysts Formulations of Comparative Examples

In this Example, the skeletal isomerization results of the zeolite comparative examples comprising crystals (Catalyst A, Example 1) v. formulations (Catalyst B, Example 2; Catalyst C, Example 3; Catalyst D, Example 4) were evaluated. The results are shown in FIG. 1, presented as average values after 100 hours on stream at 170° C. (left portion of chart) and 200° C. (right portion of chart). The Y-axis represents the percentage of C₁₄ liner olefins conversion in the feed (that is sum of yields to C₁₄ branched olefins and C₂₀₊ olefins). The area under the framed portion of each bar represents C₁₄ branched olefins yield after skeletal isomerization; the area above the framed portion of each bar represents C₂₀₊ olefins yield.

As shown, the C₂₀₊ yield decreases over formulated catalysts (Catalysts B, C, and D, respectively), and represents a lower activity, as compared to their crystal counterparts (Catalyst A), particularly at the lower temperature of 170° C. However, the overall conversion over the formulated catalysts at 170° C. is lower than their crystal counterparts. Further, steaming decreases the catalyst activity (c.f. Catalysts B and C) and upon increasing temperature from 170° C. to 200° C., the C₂₀₊ yield increases over the formulated catalysts. As shown in FIG. 1, skeletal isomerization yields at higher conversion temperatures (e.g., 200° C.) of crystal and formulated zeolite *MRE catalysts are relatively similar. The area under the framed portion of each bar represents C₁₄ branched olefins yield after skeletal isomerization; the area above the framed portion of each bar represents C₂₀₊ olefins yield. Notably, Catalyst C and D are identical, except for decreased catalyst loading and flow rate of Catalyst D, but both yield comparable C₁₄ branched and C₂₀₊ olefins.

### Example 14 (FIG. 2): Evaluation of Conventional *MRE Catalysts (Comparative Example) and Kaolin *MRE Catalysts (Experimental Examples)

In this Example, the skeletal isomerization results of the zeolite crystals of Conventional *MRE Catalysts comparative examples (Catalyst A, Example 1; Catalyst E, Example 5; Catalyst G, Example 7) v. Kaolin *MRE Catalysts experimental example (Catalyst H, Example 8) were evaluated. The results are shown in FIG. 2, presented as average values after 100 hours on stream at 170° C. (left portion of chart) and 200° C. (right portion of chart). The Y-axis represents the percentage of C₁₄ liner olefins conversion in the feed (that is sum of yields to C₁₄ branched olefins and C₂₀₊ olefins). The area under the framed portion of each bar represents C₁₄ branched olefins yield after skeletal isomerization; the area above the framed portion of each bar represents C₂₀₊ olefins yield.

As shown, the C₂₀₊ yield over the Kaolin *MRE Catalyst H example is significantly reduced, including at various Si/Al₂ ratios. The yield of C₁₄ branched olefins is ~20% less over Kaolin *MRE catalyst compared to conventional *MRE catalyst at identical Si/Al₂ ratios (c.f. Catalyst G and Catalyst H) at the lower reaction temperature of 170° C., but dramatically surpasses the sodium aluminate when the reaction temperature increases to 200° C. Indeed, at the higher reaction temperature of 200° C., the Kaolin *MRE catalyst H example is significantly greater compared to all of the comparative examples (see also FIG. 1).

### Example 15 (FIG. 3): Evaluation of Kaolin *MRE Catalysts (Experimental Examples) at Differing Si/Al₂ Ratios

In this Example, the skeletal isomerization results of Kaolin *MRE catalyst experimental examples (Catalyst H, Example 8; Catalyst I, Example 9; Catalyst J, Example 10; Catalyst K, Example 11; Catalyst L, Example 12) were evaluated to assess the influence of Si/Al₂ ratios. The results are shown in FIG. 3, presented as average values after 100 hours on stream at 170° C. (left portion of chart) and 200° C. (right portion of chart) (note that Catalyst I was not evaluated at 200° C. The Y-axis represents the percentage of C₁₄ liner olefins conversion in the feed (that is sum of yields to C₁₄ branched olefins and C₂₀₊ olefins). The framed portion of each bar represents C₁₄ branched olefins yield after skeletal isomerization; the unframed portion of each bar represents C₂₀₊ olefins yield.

As shown, the C₂₀₊ yield over the Kaolin *MRE catalysts are significantly reduced compared to their sodium aluminate counterparts (see FIG. 2), including at various Si/Al₂ ratios, demonstrating the superior performance of the Kaolin *MRE catalysts of the present disclosure. The Si/Al₂ ratio of 60 yielded slightly better results (67% C₁₄ branched olefins) in comparison, suggesting that adding more Al could improve the results of the Catalyst activity (see Catalyst G and H of FIG. 2). As shown in FIG. 3, the C₁₄ value increased significantly at the higher temperature of 200° C.

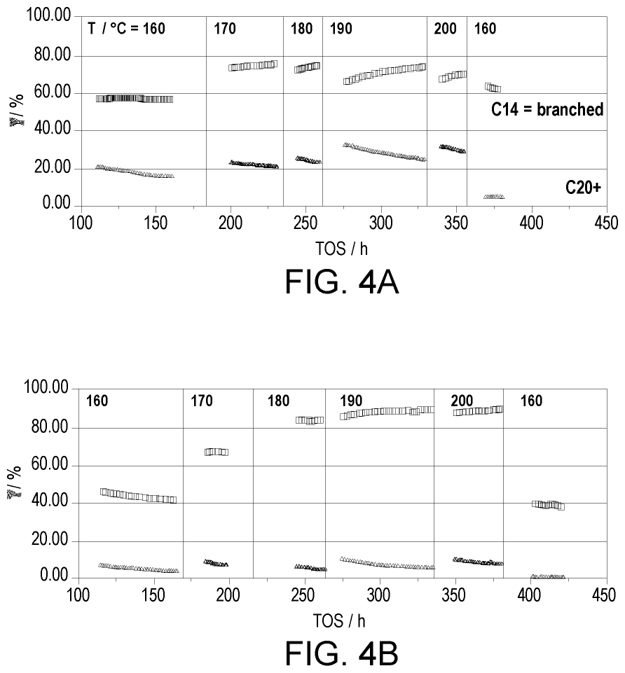

### Example 16 (FIGS. 4A and 4B): Evaluation of On-Stream Stability of Conventional *MRE Catalyst and Kaolin *MRE Catalysts (Experimental Examples)

In this Example, the on-stream stability of skeletal isomerization results of the conventional *MRE catalyst comparative example (Catalyst G, Example 7) v. Kaolin *MRE 5 catalyst experimental example (Catalyst J, Example 10) was evaluated. The results are shown in FIGS. 4A and 4B, presented at various temperatures; FIG. 4A shows the results for Catalyst G and FIG. 4B shows the results for Catalyst J. The squares represent C₁₄ branched olefins yield and the triangles represent C₂₀₊ olefins yield.

As shown in FIGS. 4A and 4B, the C₂₀₊ olefin yield decreased in increased time-on-stream, which is believed to be due to partial deactivation of sites that catalyze molecular weight growth. The C₂₀₊ yield further increased as reaction temperature is elevated. Additionally, by catalysts demonstrate relatively stable on-stream performance of C₁₄ branched olefins yield at “start-of run” (160° C.) and “end-of-run” (160° C.).

As shown in FIG. 4A, the conventional *MRE catalyst G demonstrates a relatively stable C₁₄ branched olefin yield of about 70-75%. However, as shown in FIG. 4B, the Kaolin *MRE catalyst J demonstrates a C₁₄ yield that increases with increasing temperature, reaching about 90% at 200° C. Without being bound by theory, it is believed that this elevated C₁₄ yield is due to the lower selectivity of C₂₀₊ olefins.

Accordingly, more elevated processing temperatures for the Kaolin *MRE catalysts of the present disclosure compared to conventional *MRE catalysts may be preferred, as described herein.

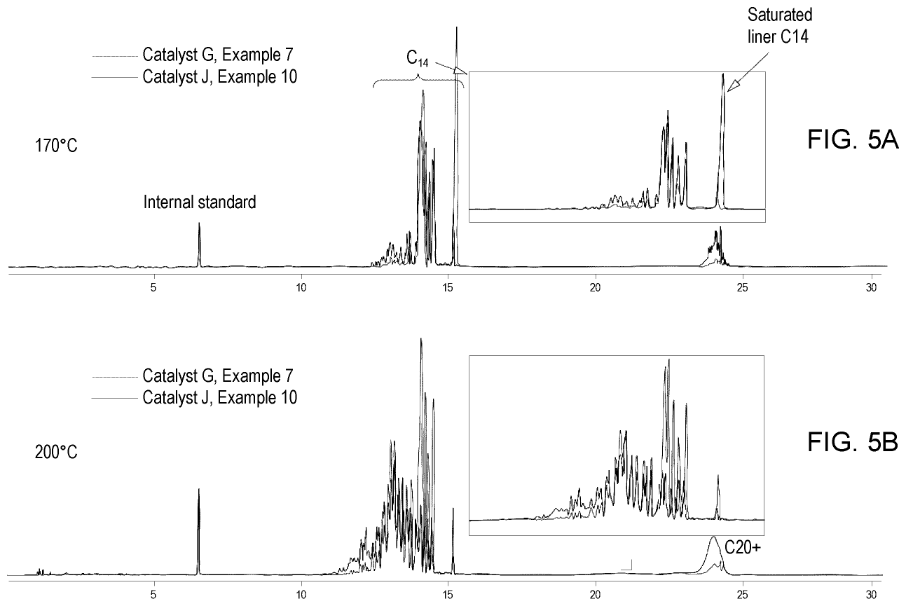

### Example 17 (FIGS. 5A and 5B): Evaluation of Olefin Chromatograms of Conventional *MRE Catalyst (Comparative Example) and Kaolin *MRE Catalyst (Experimental Examples)

In this Example, representative olefin chromatograms of skeletal isomerization results of the conventional *MRE catalyst comparative example (Catalyst G, Example 7) v. Kaolin *MRE catalyst experimental example (Catalyst J, Example 10) were evaluated. FIG. 5A represents results at 170° C. where both Catalyst G and Catalyst J yielded similar C₁₄ branched olefins yield of 67-74% in previous Examples. FIG. 5B represents results at 200° C. where both Catalyst G and Catalyst J yielded similar linear C₁₄ conversion of 97-100% in previous Examples. The zoomed in box in each of FIGS. 5A and 5B show a close-up of the C₁₄ results at each temperature. The x-axis is elapsed minutes.

As shown, at both temperatures, the Kaolin *MRE shows lower C20+ peak areas (visual confirmation based on respective chromatograms). the Kaolin *MRE Catalyst J shows a narrower C₁₄ isomer distribution and reduced C₂₀₊ yields compared to the Conventional *MRE Catalyst G. The C₁₄ yield eluting between 10-13 minutes is hindered over Kaolin *MRE as compared to the conventional *MRE catalyst comprising sodium aluminate, which without being bound by theory, is believed to be due to lower boiling point C₁₄ yield formed via excessive branching in the sodium aluminate catalyst.

Therefore, the Kaolin *MRE catalyst skeleton isomerization methods of the present disclosure are well suited for advantageously increasing C₁₄ branched olefins and decreasing C₂₀₊ olefins as described herein.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa. The term “and/or” as used in a phrase such as “A and/or B” herein is intended to include “A and B,” “A or B,” “A,” and “B.” Numerical ranges used herein include the numbers recited in the range. For example, the numerical range “from 1 wt % to 10 wt %” includes 1 wt % and 10 wt % within the recited range.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present disclosure. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

One or more illustrative embodiments are presented herein. Not all features of a physical implementation are described or shown in this application for the sake of clarity. It is understood that in the development of a physical embodiment of the present disclosure, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for one of ordinary skill in the art and having benefit of this disclosure.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein.

1. A method comprising:
contacting an olefinic feed comprising one or more C₁₀-C₂₀ alpha olefins with a Kaolin *MRE catalyst under skeletal isomerization conditions,
wherein the Kaolin *MRE catalyst is a synthesized molecular sieve having an *MRE topography, wherein in the synthesis thereof at least one source of alumina is a Kaolin family clay; and
obtaining an isomerization mixture comprising one or more C₁₀-C₂₀ branched olefins.

2. The method of claim 1, wherein the Kaolin *MRE catalyst comprises a Si:Al₂ molar ratio in the range of 15 to less than 100.

3. The method of claim 1, wherein the Kaolin *MRE catalyst comprises a Si:Al₂ molar ratio in the range of 50 to less than 100.

4. The method of claim 1, wherein the Kaolin *MRE catalyst comprises a Si:Al₂ molar ratio in the range of 60 to less than 90.

5. The method of claim 1, wherein the Kaolin family clay is selected from the group consisting of halloysite, kaolinite, dickite, nacrite, anauxite, metakaolin, and any combination thereof.

6. The method of claim 1, wherein the synthesized molecular sieve is a molecular sieve of the ZSM-48 family.

7. The method of claim 1, wherein the Kaolin *MRE catalyst is free of noble metals and transition metals in metal or oxide form.

8. The method of claim 1, wherein prior to contacting, the method further comprises forming the Kaolin *MRE catalyst into a product by combining the Kaolin *MRE catalyst with one or more of a binder, a surfactant, and/or a matrix material.

9. The method of claim 1, wherein prior to contacting, performing one or more of calcination, ion-exchange, steaming, and/or washing the Kaolin *MRE catalyst.

10. The method of claim 1, wherein the olefinic feed comprises one or more C₁₂-C₁₆ alpha olefins based on total weight of the olefinic feed.

11. The method of claim 1, wherein the olefinic feed comprises one or more C₁₂-C₁₄ alpha olefins based on total weight of the olefinic feed.

12. The method of claim 1, wherein the olefinic feed comprises at least 40 wt % of C₁₄ alpha olefins based on total weight of the olefinic feed.

13. The method of claim 1, wherein the olefinic feed comprises a branched olefin content in the range of 0 wt % to 10 wt % based on total weight of the olefinic feed.

14. The method of claim 1, wherein prior to the contacting, the method further comprises pretreating the olefinic feed reduce water content thereof to a range of 50 ppmw water based on the weight of the olefinic feed.

15. The method of claim 1, wherein the skeletal isomerization conditions comprise a temperature in the range of about 160° C. to about 300° C.

16. The method of claim 1, wherein the skeletal isomerization conditions comprise a pressure in the range of 0 barg to 2 barg.

17. The method of claim 1, wherein the skeletal isomerization conditions comprise contacting the olefin feed at a weight hourly space velocity in the range of from 1 h-1 to about 20 h-1 based on weight of the olefin feed flowing per unit of weight of the Kaolin *MRE catalyst per hour.

18. The method of claim 1, wherein the conversion of the C₁₀-C₂₀ alpha olefins is from about 20% to about 98%.

19. The method of claim 1, wherein the isomerization mixture comprises less than 20 wt % of C₂₀₊ olefins based on total weight of the isomerization mixture.

20. The method of claim 1, wherein the isomerization mixture comprises 9 wt % to 90 wt % of linear internal olefins based on total weight of the isomerization mixture.
